SEQUENCE LISTING (11 sequences)

SEQ ID NO: 1 - DNA, 2232 bp, Homo sapiens
  source 1..2232: mol_type = genomic DNA
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactccttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattaccccggctgggtggccggctcttggtga

SEQ ID NO: 2 - protein, 743 aa, Homo sapiens
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPGWVAGSW

SEQ ID NO: 3 - DNA, 1509 bp, Homo sapiens
  source 1..1509: mol_type = genomic DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 4 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 5 - DNA, 3878 bp, Homo sapiens
  source 1..3878: mol_type = genomic DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggaattcagaccatggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactccttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattaccccggctgggtggccggctcttggtgatagattcgccaccactgggccttgttttccgctaattccagggcagattccagggcccagagctggacagacatcacaggataacccaggcctgggaggaggccccacggcctgctggtggggtctgacctggggggattggagggaaatgacctgccctccaccaccacccaaagtgtgggattggtaccgagctcggatccactagtcccgggccatcacactgggaattggccgcgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccc

SEQ ID NO: 6 - DNA, 20 bp, synthetic construct
  misc_feature 1..20: note = Synthetic primer
  source 1..20: mol_type = other DNA
ggcagtacatcaagtgtatc

SEQ ID NO: 7 - DNA, 21 bp, synthetic construct
  misc_feature 1..21: note = Synthetic primer
  source 1..21: mol_type = other DNA
accaatggtaatagcgatgac

SEQ ID NO: 8 - DNA, 20 bp, synthetic construct
  misc_feature 1..20: note = Synthetic probe
  source 1..20: mol_type = other DNA
aatgacggtaaatggcccgc

SEQ ID NO: 9 - DNA, 20 bp, synthetic construct
  misc_feature 1..20: note = Synthetic primer
  source 1..20: mol_type = other DNA
gtcatcactattggcaacga

SEQ ID NO: 10 - DNA, 20 bp, synthetic construct
  misc_feature 1..20: note = Synthetic primer
  source 1..20: mol_type = other DNA
ctcaggagttttgtcacctt

SEQ ID NO: 11 - DNA, 20 bp, synthetic construct
  misc_feature 1..20: note = Synthetic probe
  source 1..20: mol_type = other DNA
ttccgatgccctgaggctct

(19) United States
(12) Patent Application Publication
(10) Pub. No.: US 2025/0312423 A1
(43) Pub. Date: Oct. 9, 2025

(54) Methods for Delivery of Polynucleotides by Adeno-Associated Virus for Lysosomal Storage Disorders

(71) Applicant: NATIONWIDE CHILDREN'S HOSPITAL, INC., Columbus, OH (US)

(72) Inventors: Douglas M. McCarty, Pataskala, OH (US); Haiyan Fu, Pataskala, OH (US)

(21) Appl. No.: 19/202,761

(22) Filed: May 8, 2025

Related U.S. Application Data
(63) Continuation of application No. 15/903,861, filed on Feb. 23, 2018, now Pat. No. 12,324,829.
Application No. 15/903,861 is a continuation of application No. 14/950,387, filed on Nov. 24, 2015, now abandoned.
Application No. 14/950,387 is a continuation of application No. 13/922,915, filed on Jun. 20, 2013, now abandoned.
Application No. 13/922,915 is a division of application No. 13/491,326, filed on Jun. 7, 2012, now abandoned.
(60) Provisional application No. 61/494,635, filed on Jun. 8, 2011.

(51) Int. Cl. A61K 38/46; A61K 35/761; A61K 47/26; A61K 48/00; C12N 7/00; C12N 9/14; C12N 15/86

(52) U.S. Cl. CPC A61K 38/46; A61K 35/761; A61K 47/26; A61K 48/005; C12N 7/00; C12N 9/14; C12N 15/86; C12N 2750/14121; C12N 2750/14143; C12N 2750/14171; C12Y 310/01001

(57) ABSTRACT

The present invention relates to methods and materials useful for systemically delivering polynucleotides across the blood brain barrier using adeno-associated virus as a vector. For example, the present invention relates to methods and materials useful for systemically delivering α-N-acetylglucosamidinase polynucleotides to the central and peripheral nervous systems, as well as the somatic system. Use of these methods and materials is indicated, for example, for treatment of the lysosomal storage disorder mucopolysaccharidosis IIIB. As another example, the present invention relates to methods and materials useful for systemically delivering N-sulphoglucosamine sulfphohydrolase polynucleotides to the central and peripheral nervous systems, as well as the somatic system. Use of this second type of methods and materials is indicated, for example, for treatment of the lysosomal storage disorder mucopolysaccharidosis IIIA.

This application claims the benefit of the filing date of U.S. Provisional Patent Application No. 61/494,635 filed Jun. 8, 2011, which is incorporated by reference herein in its entirety.

### FIELD OF THE INVENTION

The present invention relates to methods and materials useful for systemically delivering polynucleotides across the blood brain barrier using adeno-associated virus as a vector. For example, the present invention relates to methods and materials useful for systemically delivering α-N-acetylglucosamidinase polynucleotides to the central and peripheral nervous systems, as well as the somatic system. Use of these methods and materials is indicated, for example, for treatment of the lysosomal storage disorder mucopolysaccharidosis IIIB. As another example, the present invention relates to methods and materials useful for systemically delivering N-sulphoglucosamine sulfphohydrolase polynucleotides to the central and peripheral nervous systems, as well as the somatic system. Use of this second type of methods and materials is indicated, for example, for treatment of the lysosomal storage disorder mucopolysaccharidosis IIIA.

### Incorporation by Reference of the Sequence Listing

This application contains, as a separate part of disclosure, a Sequence Listing in computer-readable form (46031A_SeqListing.txt; 22,737 byte ASCII text file, created Jun. 7, 2012) which is incorporated by reference herein in its entirety.

### BACKGROUND

Mucopolysaccharidosis (MPS) IIIB is a devastating lysosomal storage disease (LSD) caused by autosomal recessive defects in the gene coding a lysosomal enzyme, α-N-Acetylglucosaminidase (NAGLU). The lack of NAGLU activity disrupts the stepwise degradation of a class of biologically important glycosaminoglycan (GAG), leading to the accumulation of heparan sulfate oligosaccharides in lysosomes in cells of most tissues. Cells throughout the CNS are particularly affected, resulting in complex secondary neuropathology. MPS IIIB infants appear normal at birth, but develop progressive neurological manifestations that lead to premature death. Somatic manifestations of MPS IIIB occur in all patients, and involve virtually all organs, although they are mild relative to other forms of MPS, such as MPS I, II and VII.

MPS IIIA is a related LSD caused by autosomal recessive defects in the gene encoding a lysosomal enzyme, N-sulphoglucosamine sulphohydrolase (SGSH). The lack of SGSH activity also disrupts the stepwise degradation of a class of biologically important GAG, leading to the accumulation of heparin sulfate oligosaccharides in lysosomes in cells of most tissues.

No treatment is currently available for MPS IIIB or IIIA. For all of the MPS disorders, therapies have historically been limited to supportive care and management of complications. MPS IIIB is not amenable to either hematopoietic stem cell transplantation or recombinant enzyme replacement therapy. These have instead been used to treat mostly somatic disorders in patients with MPS I, II and IV. This is because the neuropathology of MPS IIIB is global and the blood brain barrier (BBB) precludes effective central nervous system (CNS) access.

For the majority of CNS diseases, effective treatments are rare since the CNS is located in a well protected environment and isolated by a highly defined anatomical/functional barrier. The BBB is completely formed at birth in humans. In general, the BBB protects the CNS by selectively regulating the transport of molecules/agents from the blood circulation into the CNS or vice versa. Likewise, it prevents potential therapeutics from entering the CNS. The BBB remains the most critical challenge to developing therapies for CNS diseases, especially global CNS disorders.

It is contemplated herein that gene therapy has potential for treating LSDs because the secretion of lysosomal enzymes, including NAGLU and SGSH, leads to bystander effects thus reducing the demand for gene transfer efficiency. The adeno-associated virus (AAV) vector system is one system with demonstrated therapeutic effect in a great variety of disease models. To date, no known pathogenesis has been linked to AAV in humans. Recombinant AAV (rAAV) vectors based on AAV serotype 2 (AAV2) have been used in numerous studies for neurological diseases, transducing both neuronal and non-neuronal cells in the CNS with demonstrated therapeutic benefits in treating MPS and other LSDs in animals and in patients with Parkinson's and Batten's disease. In the majority of rAAV-CNS gene therapy studies in LSDs, vectors were delivered by direct intracranial injection, which has limited potential for treating global CNS diseases. See, Sands et al., Acta Paediatr. Suppl., 97: 22-27 (2008); Fu et al., Mol, Ther., 5: 42-49 (2002); Cressant et al., J. Neurosci., 24: 10229-10239 (2004); Fraldi et al., Hum. Mol. Genet., 16: 2693-2702 (2007); Worgall et al., Hum. Gen. Ther., 19: 563-574 (2008) and Heldermon et al. Mol. Ther., 18: 873-880 (2010). To overcome these obstacles, more efficient delivery approaches have been developed with broad or global transduction, and functional benefits for the neurological disease in adult MPS IIIB mice. An intracisternal injection of rAAV2-hNAGLU vector in adult MPS IIIB mice, following mannitol pretreatment, led to deep periventricular transduction and clinical benefits. See Fu et al., J. Gene Med., 12: 624-633 (2010). Intravenous (IV) rAAV injection into neonatal MPS I, MPS VII and MPS IIIB mice led to long-term correction of lysosomal storage in both somatic and CNS tissues. See, Sands et al., Lab. Anim. Sci., 49: 328-330 (1999); Hartung et al., Mol. Ther., 9: 866-875 (2004) and Heldermon et al., supra. However, the BBB may still be permeable in neonatal mice while closed at birth in humans. Previously, in adult MPS IIIB mice, pretreatment with an N infusion of mannitol transiently disrupting the BBB facilitated the CNS entry of IV-delivered rAAV2, resulting in diffuse global CNS transduction and neurological correction. See, McCarty et al., Gene Ther., 16: 1340-1352 (2009).

Recombinant AAV9 vectors encoding the sulfamidase enzyme have been administered to MPSIIIA mice as reported in Ruzo et al., XVII Annual Congress of the European Society of Gene and Cell Therapy: 1389 (Abstract Or 96) (October 2010) and Ruzo et al., Mol. Therap., 20(2): 254-266 (2012).

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). Cis-acting sequences directing viral DNA replication, encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p¹9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus, making cold preservation of AAV less critical. AAV may even be lyophilized. Finally, AAV-infected cells are not resistant to superinfection.

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11. AAV9 is described in U.S. Pat. No. 7,198,951 and in Gao et al., J. Virol., 78: 6381-6388 (2004). Advances in the delivery of AAV6 and AAV8 have made possible the transduction by these serotypes of skeletal and cardiac muscle following simple systemic intravenous or intraperitoneal injections. See, Pacak et al., Circ. Res., 99(4): 3-9 (1006) and Wang et al., Nature Biotech., 23(3): 321-328 (2005). The use of some serotypes of AAV to target cell types within the central nervous system, though, has required surgical intraparenchymal injection. See, Kaplitt et al., Lancet 369: 2097-2105 (2007); Marks et al., Lancet Neurol 7: 400-408 (2008); and Worgall et al., Hum Gene Ther (2008).

There remains a need in the art for products and methods for treating lysosomal storage disorders such as MPS IIIB and MPS IIIA.

### SUMMARY

The present invention provides methods and materials useful for systemically delivering polynucleotides such as NAGLU polynucleotides or SGSH polynucleotides across the BBB.

According to the invention, gene delivery is achieved by utilizing, for example, AAV serotype 9 (AAV9). See, Foust et al., Nature Biotechnology, 27: 59-65 (2009); Duque et al., Mol. Ther. 17: 1187-1196 (2009); and Zincarelli et al., Mol. Ther., 16: 1073-1080 (2008). Vectors based on this serotype or functionally-related serotypes are able to cross the BBB unaided in neonate and adult animals. An added benefit to using AAV9 vectors is that pre-existing immunity is less common than for AAV2 serotype. The use of rh74 serotype AAV vectors among others is also contemplated by the invention.

In one aspect, the invention provides a method of delivering a NAGLU polynucleotide across the BBB comprising systemically administering a rAAV9 with a genome including the polynucleotide to a patient. In some embodiments the rAAV9 genome is a single-stranded genome.

More specifically, the present invention provides methods and materials useful for systemically delivering NAGLU polynucleotides across the blood brain barrier to the central and peripheral nervous system. In some embodiments, a method is provided of delivering a polynucleotide to the central nervous system comprising systemically administering a rAAV9 with a single-stranded genome including the genome to a patient. In some embodiments, a method of delivering a NAGLU polynucleotide to the peripheral nervous system comprising systemically administering a rAAV9 with a single-stranded genome including the polynucleotide to a patient is provided.

Even more specifically, in some embodiments, the NAGLU polynucleotide is delivered to brain. In some embodiments, the polynucleotide is delivered to the spinal cord. In some embodiments, the NAGLU polynucleotide is delivered to a lower motor neuron. In some embodiments, the polynucleotide is delivered to nerve and glial cells. In some embodiments, the glial cell is a microglial cell, an oligodendrocyte or an astrocyte. In some, embodiments, the rAAV9 is used to deliver a NAGLU polynucleotide to a Schwann cell.

Use of the NAGLU methods and materials is indicated, for example, for treating Sanfilippo syndrome Type B/MPS IIIB.

In another aspect, the invention provides a method of delivering an SGSH polynucleotide across the BBB comprising systemically administering a rAAV9 with a genome including the polynucleotide to a patient. In some embodiments, the rAAV9 genome is a self-complementary genome. In some embodiments the rAAV9 genome is a single-stranded genome.

More specifically, the present invention provides methods and materials useful for systemically delivering SGSH polynucleotides across the blood brain barrier to the central and peripheral nervous system. In some embodiments, a method is provided of delivering a polynucleotide to the central nervous system comprising systemically administering a rAAV9 with a self-complementary genome including the genome to a patient. In some embodiments, a method of delivering a SGSH polynucleotide to the peripheral nervous system comprising systemically administering a rAAV9 with a self-complementary genome including the polynucleotide to a patient is provided.

Even more specifically, in some embodiments, the SGSH polynucleotide is delivered to brain. In some embodiments, the polynucleotide is delivered to the spinal cord. In some embodiments, the SGSH polynucleotide is delivered to a lower motor neuron. In some embodiments, the polynucleotides is delivered to nerve and glial cells. In some embodiments, the glial cell is a microglial cell, an oligodendrocyte or an astrocyte. In some, embodiments, the rAAV9 is used to deliver a SGSH polynucleotide to a Schwann cell.

Use of the SGSH methods and materials is indicated, for example, for treating MPS IIIA.

In yet another aspect, administration of the rAAV9 encoding a NAGLU or SGSH polypeptide is preceded by administration of mannitol.

In still another aspect, the invention provides rAAV genomes comprising one or more AAV ITRs flanking a polynucleotide encoding a NAGLU. The NAGLU polynucleotide is operatively linked to transcriptional control DNAs, specifically promoter DNA and polyadenylation signal sequence DNA that are functional in target cells to form a gene cassette. The gene cassette may also include intron sequences to facilitate processing of an RNA transcript when expressed in mammalian cells.

In a further aspect, the invention provides rAAV genomes comprising one or more AAV ITRs flanking a polynucleotide encoding an SGSH. The SGSH polynucleotide is operatively linked to transcriptional control DNAs, specifically promoter DNA and polyadenylation signal sequence DNA that are functional in target cells to form a gene cassette. The gene cassette may also include intron sequences to facilitate processing of an RNA transcript when expressed in mammalian cells.

The rAAV genomes of the invention lack AAV rep and cap DNA. AAV DNA in the rAAV genomes (e.g., ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004).

NAGLU polypeptides contemplated include, but are not limited to, a NAGLU polypeptide with the amino acid sequence set out in SEQ ID NO: 2.

SGSH polypeptides contemplated include, but are not limited to, a SGSH polypeptide with the amino acid sequence set out in SEQ ID NO: 4.

The polypeptides contemplated include full-length proteins, precursors of full length proteins, biologically active subunits or fragments of full length proteins, as well as biologically active analogs (e.g., derivatives and variants) of any of these forms of polypeptides. Thus, polypeptides include, for example, those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a nucleic acid or an amino acid sequence described herein.

As used herein “biologically active derivative,” “biologically active fragment,” “biologically active analog” or “biologically active variant” includes any derivative or fragment or analog or variant of a molecule having substantially the same functional and/or biological properties of said molecule, such as enzymatic activities.

An “analog,” such as a “variant” or a “derivative,” is a compound substantially similar in structure to and having the same biological activity as, albeit in certain instances to a differing degree, a naturally-occurring molecule.

A “derivative,” for example, is a type of analog and refers to a polypeptide sharing the same or substantially similar structure as a reference polypeptide that has been modified, e.g., chemically.

A polypeptide variant, for example, is a type of analog and refers to a polypeptide sharing substantially similar structure and having the same biological activity as a reference polypeptide (i.e., “native polypeptide” or “native therapeutic protein”). Variants differ in the composition of their amino acid sequences compared to the naturally-occurring polypeptide from which the variant is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence.

Variant polypeptides include insertion variants, wherein one or more amino acid residues are added to a therapeutic protein amino acid sequence of the present disclosure. Insertions may be located at either or both termini of the protein, and/or may be positioned within internal regions of the therapeutic protein amino acid sequence. Insertion variants, with additional residues at either or both termini, include for example, fusion proteins and proteins including amino acid tags or other amino acid labels.

In deletion variants, one or more amino acid residues in a therapeutic protein polypeptide as described herein are removed. Deletions can be effected at one or both termini of the therapeutic protein polypeptide, and/or with removal of one or more residues within the therapeutic protein amino acid sequence. Deletion variants, therefore, include fragments of a polypeptide sequence.

In substitution variants, one or more amino acid residues of a therapeutic protein polypeptide are removed and replaced with alternative residues. In one aspect, the substitutions are conservative in nature and conservative substitutions of this type are well known in the art. Alternatively, the present disclosure embraces substitutions that are also non-conservative. Exemplary conservative substitutions are described in Lehninger, [Biochemistry, 2nd Edition; Worth Publishers, Inc., New York (1975), pp.71-77] and are set out immediately below.

CONSERVATIVE SUBSTITUTIONS

| SIDE CHAIN | |
|---|---|
| CHARACTERISTIC | AMINO ACID |
| Non-polar (hydrophobic): | |
| A. Aliphatic | A L I V P |
| B. Aromatic | F W |
| C. Sulfur-containing | M |
| D. Borderline | G |
| Uncharged-polar: | |
| A. Hydroxyl | S T Y |
| B. Amides | N Q |
| C. Sulfhydryl | C |
| D. Borderline | G |
| Positively charged (basic) | K R H |
| Negatively charged (acidic) | D E |

Alternatively, exemplary conservative substitutions are set out immediately below.

| CONSERVATIVE SUBSTITUTIONS II | |
|---|---|
| ORIGINAL | EXEMPLARY |
| RESIDUE | SUBSTITUTION |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

In yet further aspect, the invention provides DNA plasmids comprising rAAV genomes of the invention. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10 and AAV-11. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In another aspect, the invention provides rAAV (i.e., infectious encapsidated rAAV particles) comprising a rAAV genome of the invention. In some embodiments of the invention, the rAAV genome is a self-complementary genome.

The invention includes, but is not limited to, the exemplified rAAV named “rAAV9-CMV-hNAGLU.” The rAAV genome has in sequence an AAV2 ITR, the cytomegalovirus (CMV) immediate early promoter/enhancer, an SV40 intron (SD/SA), the NAGLU DNA set out in SEQ ID NO: 1, a polyadenylation signal sequence from bovine growth hormone and another AAV2 ITR. The DNA sequence of the vector genome is set out in SEQ ID NO: 5. The genome lacks AAV rep and cap DNA, that is, there is no AAV rep or cap DNA between the ITRs of the genome.

The invention also includes, but is not limited to, rAAV encoding SGSH. In some embodiments, the rAAV genome has in sequence an AAV2 ITR, the CMV immediate early promoter/enhancer, the SGSH DNA set out in SEQ ID NO: 3, a polyadenylation signal sequence from bovine growth hormone and a AAV2 ITR lacking the terminal resolution site. In some embodiments, the rAAV genome has in sequence an AAV2 ITR, the mouse Ula promoter, the SGSH DNA set out in SEQ ID NO: 3, a polyadenylation signal sequence from bovine growth hormone and a AAV2 ITR lacking the terminal resolution site. In some embodiments, rAAV genome has in sequence an AAV2 ITR, the mouse Ula promoter, an intron, the SGSH DNA set out in SEQ ID NO: 3, a polyadenylation signal sequence from bovine growth hormone and a AAV2 ITR lacking the terminal resolution site. The genomes lack AAV rep and cap DNA, that is, there is no AAV rep or cap DNA between the ITRs of the genomes.

NAGLU and SGSH DNAs include, without limitation, those that (1) hybridize under stringent hybridization conditions to a nucleic acid encoding an amino acid sequence as described herein, and conservatively modified variants thereof; (2) have a nucleic acid sequence that has greater than about 95%, about 96%, about 97%, about 98%, about 99%, or higher nucleotide sequence identity, over a region of at least about 25, about 50, about 100, about 150, about 200, about 250, about 500, about 1000, or more nucleotides (up to the full length sequence of the mature protein), to a nucleic acid sequence as described herein. Exemplary “stringent hybridization” conditions include hybridization at 42° C. in 50% formamide, 5×SSC, 20 mM Na·PO4, pH 6.8; and washing in 1×SSC at 55° C. for 30 minutes. It is understood that variation in these exemplary conditions can be made based on the length and GC nucleotide content of the sequences to be hybridized. Formulas standard in the art are appropriate for determining appropriate hybridization conditions. See Sambrook et al., Molecular Cloning: A Laboratory Manual (Second ed., Cold Spring Harbor Laboratory Press, 1989) §§ 9.47-9.51.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In an additional aspect, the invention contemplates compositions comprising rAAV of the present invention encoding an NAGLU polypeptide. These compositions may be used to treat mucopolysaccharidosis IIIB. In other embodiments, compositions of the present invention may include two or more rAAV encoding different polypeptides of interest.

In still an additional aspect, the invention contemplates compositions comprising rAAV of the present invention encoding an SGSH polypeptide. These compositions may be used to treat mucopolysaccharidosis IIIA. In other embodiments, compositions of the present invention may include two or more rAAV encoding different polypeptides of interest.

Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰ about 1×10¹¹, about 1×10¹², about 1×10¹³ to about 1×10¹⁴ or more DNase resistant particles (DRP) per ml. Dosages may also be expressed in units of viral genomes (vg). Dosages may also vary based on the timing of the administration to a human. These dosages of rAAV may range from about 1×10¹¹ vg/kg, about 1×10¹², about 1×10¹³, about 1×10¹⁴, about 1×10¹⁵, about 1×10¹⁶ or more viral genomes per kilogram body weight in an adult. For a neonate, the dosages of rAAV may range from about 1×10¹¹, about 1×10¹², about 3×10¹², about 1×10¹³, about 3×10¹³, about 1×10¹⁴, about 3 x10¹⁴, about 1×10¹⁵, about 3 x10¹⁶, about 1×10¹⁶, about 3 x10¹⁶ or more viral genomes per kilogram body weight.

Treatment by methods of the invention comprises the step of administering an intravenous (IV) effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

Combination therapies are also contemplated by the invention. Combination as used herein includes both simultaneous treatment or sequential treatments. Combinations of methods of the invention with standard medical treatments (e.g., transient or long-term immunosuppression) are specifically contemplated, as are combinations with novel therapies.

Compositions suitable for systemic (IV) use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin, and Tween family of products (e.g., Tween 20).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction of cells with rAAV of the invention results in sustained expression of NAGLU or SGSH polypeptide. Transduction may be carried out with gene cassettes comprising tissue specific control elements, for example, promoters that allow expression specifically within neurons or specifically within astrocytes. Examples include neuron specific enolase and glial fibrillary acidic protein promoters. Inducible promoters under the control of an ingested drug may also be developed.

It will be understood by one of ordinary skill in the art that a polynucleotide delivered using the materials and methods of the invention can be placed under regulatory control using systems known in the art. By way of non-limiting example, it is understood that systems such as the tetracycline (TET on/off) system [see, for example, Urlinger et al., Proc. Natl. Acad. Sci. USA 97(14):7963-7968 (2000) for recent improvements to the TET system] and Ecdysone receptor regulatable system [Palli et al., Eur J. Biochem 270: 1308-1315 (2003] may be utilized to provide inducible polynucleotide expression.

Thus, the invention provides methods of systemically administering an effective dose (or doses, administered essentially simultaneously or doses given at intervals) of rAAV of the invention to a patient in need thereof.

### BRIEF DESCRIPTION OF THE DRAWING

FIG. 1 is a map of the rAAV-CMV-hNAGLU vector genome.

FIG. 2 shows improved behavior and extended survival in MPS IIIB mice after systemic gene transfer by rAAV-CMV-hNAGLU. FIG. 2a. Hidden task in water maze (n=11/group). Day 1: test trial. FIG. 2b. Latency to fall from a rotarod (n=11/group). FIG. 2c. Survival (i 5/group, P<0.001). +/+: wt; −/−: MPS IIIB; AAV9-L, AAV9-H: MPS IIIB mice treated with 5×10¹² or 1.5×10¹³ vg/kg rAAV9-hNAGLU vector, respectively. *: P<0.05 (vs. +/+); #: P<0.05 (vs. AAV9-L); \hat{ }: P<0.05 (vs. AAV9-H); &: P>0.05 (vs. −/−). Repeated measures ANOVA analyses: @: day effect P<0.01; $: group (treatment effect) P<0.01; %: day-group interaction P41.020 (rotarod).

FIG. 3 shows rAAV9-mediated expression of functional rNAGLU in tissues. Tissues from MPS IIIB mice treated with rAAV9-hNAGLU were assayed for NAGLU activity (6 and 9mo pi)(n=5-6/group). FIG. 3a. Dose-response. +/+: wt; AAV9-11, AAV9-L: MPS IIIB mice treated with 1.5×10¹³ (AAV9-H) or 5×10¹² vg/kg (AAV9-L) vector; FIG. 3b. Impact of mannitol pretreatment. M+/M−: MPS IIIB mice treated with 2×10¹³ vg/kg vector with (M+) or without (M−) mannitol pretreatment. FIG. 3c. Plasma NAGLU activity (n=3-4). +/−: heterozygotes. No significant difference in tissue NAGLU activity was detected at 6 and 9 months pi. Data shown are means±SD of combined data on tissues from mice at 6 and 9mo pi. *: P<0.01 vs. +/+; #: P<0.05 vs. AAV9-H or M+: P>0.05 vs. +/+. @: P<0.05 vs. +/−.

FIG. 4 shows the significant reduction of GAG content in the CNS and somatic tissues. Tissues from MPS IIIB mice treated with rAAV9-hNAGLU were assayed to quantify GAG content (6 and 9mo pi). FIG. 4a. Dose response. FIG. 4b. Impact of mannitol pretreatment. +/Ai wt; −/−: MPS IIIB; AAV9-H, AAV9-L: MPS MB mice treated with 1.5 x1013 vg or 5 x1012 vg/kg vector; M+, M−: MPS IIIB mice treated with rAAV9 vector (2 x1013 vg/kg) with or without mannitol pretreatment. Data shown are means±SD (n=5-6), combining data from tissues collected at 6 and 9mo pi. *: P<0.01 vs. +/+; #: P<0.05 vs. AAV9-H or M+; \hat{ }: P<0.05 vs. AAV9-L or M−; +: P>0.05 vs. +/+.

FIG. 5 shows rAAV9-mediated correction of astrocytosis and neurodegeneration in MPS IIIB mice. Brain sections of MPS IIIB mice treated with rAAV9-CMV-hNAGLU vector (6mo pi) were assayed for GFAP by immunofluorescence and stained with toluidine blue for histopathology. FIG. 5a. Number of astrocytes: Data are means±SD of GFAP+ cells per 330 x433 pm on 6-8 IF-GFAP-staining sections/mouse, from 3 mice/group. FIG. 5b. Number of purkinje cells: Data are means±SD of purkinje cells/200p.m (in length) in ansiform lobules in cerebellum on 6 toluidine blue stained sections/mouse, from 3 mice/group. NT: non-treated MPS IIIB mouse; AAV9: MPS IIIB mouse treated with rAAV9. CTX: cerebral cortex; ST: Striatum; TH: thalamus; BS: Brain stem. *: P<0.01 vs. non-treated.

FIG. 6 shows rAAV9-mediated expression of functional rSGSH in tissues of treated MPSIIIA mice. For each tissue, AAV9, rh74 and untreated result bars are respectively shown from left to right.

FIG. 7 shows a significant reduction of GAG content in tissues of treated MPSIIIA mice. For each tissue, AAV9, rh74 and untreated result bars are respectively shown from left to right.

FIG. 8 shows an improvement in cognitive behavior assays after treatment of one-month old MPSIIIA mice with low dose scAAV9 or rh74-U1a-SGSH. In the lower graphs, for each tissue, untreated, wild type and either AAV9 or rh74 result bars are respectively shown from left to right.

FIG. 9 shows a significant reduction of GAG content in tissues of MPSIIIA mice treated at 2 or 6 months of age. For each tissue, wild type, untreated, two-month and six-month result bars are respectively shown from left to right.

FIG. 10 shows an improvement in cognitive behavior assays after treatment of two- or six-month old MPSIIIA mice with high dose scAAV9 or rh74-U1a-SGSH. In the lower graphs, for each tissue, untreated, wild type and either AAV9 or rh74 result bars are respectively shown from left to right.

### DETAILED DESCRIPTION

The present invention is illustrated by the following examples relating to delivery of human NAGLU (hNAGLU) genes to the spinal cord via intravenous delivery of rAAV9. Example 1 describes rAAV encoding hNAGLU. Example 2 describes the administration of the rAAV encoding hNAGLU to MPSIIIB mice. Examples 3 through 6 describe the beneficial results of administration of the rAAV. Example 7 discusses the significance of the results. Example 8 describes rAAV encoding SGSH. Examples 9 through 11 describe administration of various dosages of rAAV encoding SGSH to MPSIIIA mice of varying ages, as well as the beneficial effects of the administration.

### Example 1

### Recombinant AAV (rAAV) Viral Vectors Encoding NAGLU

A rAAV vector plasmid, containing AAV2 ITRs, an immediate early CMV promoter/enhancer, an SV40 intron, a human α-N-acetylglucosaminidase coding region, a bGH polyadenylation signal sequence, and ampicillin resistance gene, was used to produce a rAAV9-CMV-hNAGLU viral vector.

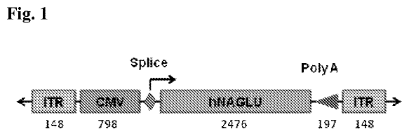

Recombinant AAV9 viral vectors with the hNAGLU-encoding genome were produced in 293 cells using three-plasmid co-transfection, and purified as described in Zolotukhin et al., Gene Ther., 6: 973-985 (1999). This vector is referred to as “rAAV9-CMV-hNAGLU” herein. The vector genomes contained minimal elements required for transgene expression, including AAV2 terminal repeats, a human cytomegalovirus (CMV) immediate-early promoter, SV40 splice donor/acceptor signal, a human NAGLU coding sequence (SEQ ID NO: 1), and bGH polyadenylation signal. SEQ ID NO: 5 is the DNA sequence of the vector genome. FIG. 1 is a map of the vector genome wherein the length of the various elements of the genome is indicated below the element.

A control self-complementary AAV encoding green fluorescent protein, scAAV9-CMV-GFP was also produced, containing AAV2 terminal repeats, a human cytomegalovirus (CMV) immediate-early promoter, SV40 splice donor/acceptor signal, a eGFP coding sequence, and SV40 polyadenylation signal.

### Example 2

### Administration of Viral Vectors

An MPS IIB3 knock-out mouse colony [Li et al., Proc. Natd. Acad. Sci. USA, 96: 14505-14510 (1999) was maintained on an inbred background (C57BL/6) of backcrosses of heterozygotes. All care and procedures were in accordance with the Guide for the Care and Use of Laboratory Animals [DHHS Publication No. (NIH) 85-23]. The genotypes of progeny mice were identified by PCR.

To assess the therapeutic efficacy of rAAV9 gene delivery, 4-6-week-old MPS IIIB mice were treated with an IV injection of rAAV9-CMV-hNAGLU (5×10¹² or 1.5×10¹³ vg/kg, n=11/group). Separately, other MPS IIIB mice were treated with 2×10¹³ vg/kg rAAV9-CMV-hNAGLU, with or without mannitol pretreatment (n=5/group), to assess the impact on CNS entry. Controls were sham-treated (phosphate-buffered saline) wild type (wt) and MPS IIIB littermates (n=11). Tissue analyses were carried out at 6 months and 9 months (n=2-4/group) post-injection (pi).

Additionally, self-complementary AAV (scAAV) vector carrying a cytomegalovirus-green fluorescent protein (CMV-GFP) transgene (5×10¹² vg/kg) was injected IV into 6-8-week-old wt mice (n=4/group) to determine the distribution of transgene expression (1 month pi), as a comparison to rAAV9-hNaGlu treatment.

Results are presented below.

### Example 3

### Behavioral Tests

The rAAV9-CMV-hNaGlu-treated MPS BIB mice and controls were tested for behavioral performance at approximately 5.0-5.5 months of age as follows.

### Hidden Task in the Morris Water Maze

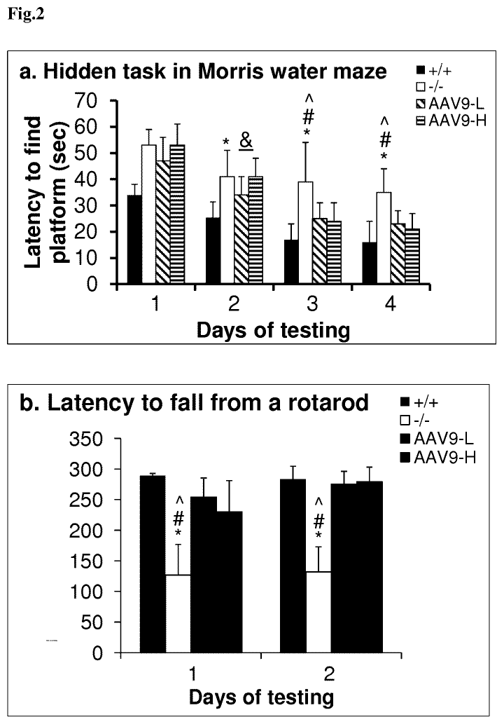

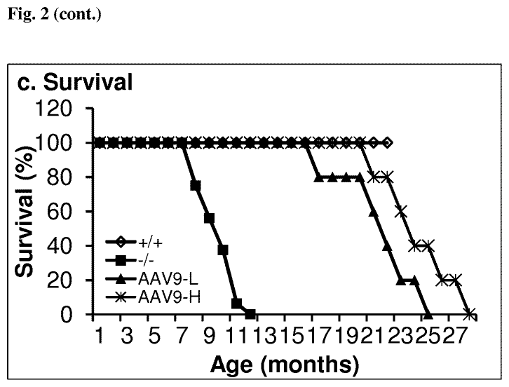

A water maze (diameter-122 cm) was filled with water (45 cm deep, 24-26° C.) containing 1% white TEMPERA paint, located in a room with numerous visual cues. See Warburton et al., J. Neurosci, 21: 7323-7330 (2001). Mice were tested for their ability to find a hidden escape platform (20×20 cm) 0.5 cm under the water surface. Each animal was given four trials per day, across three days, as described previously. Measures were taken of latency to find the platform (sec) via an automated tracking system (San Diego Instruments). Results are shown in FIG. 2a.

### Rotarod

Mice were tested on an accelerating rotarod (Med Associate, Inc.) to assess motor coordination. See Lijam et al., Cell, 90895-905 (1997). Rotation speed was set at an initial value of 3 revolutions per minute (rpm), with a progressive increase to a maximum of 30 rpm across five minutes (the maximum trial length). For the first test session, animals were given three trials, with 45 seconds between each trial. Two additional trials were given 48 hours later. Measures were taken for latency to fall from the top of the rotating barrel. Results are shown in FIG. 2b.

### Statistical Analyses

Means, standard deviation (SD) and unpaired student t-test were used to analyze quantitative data. Behavioral measures were taken by an observer blind to experimental treatment. Behaviroal testing data were also analyzed using repeated measures ANOVA (SAS 9.1.3) to determine the significance of the variances among treatment and control groups and testing days.

### Results of Behavioral Tests

All mice treated IV with 5×10¹² or 1.5×10¹³ vg/kg rAAV9-NAGLU were tested for behavior at 5-5.5 mo of age to assess the neurological impacts. Both dosage groups exhibited significant decreases in latency to find a hidden platform in a water maze (FIG. 2a), and significantly longer latency to fall from an accelerating rotarod (FIG. 2b), compared with non-treated MPS IIIB mice, indicating the correction of cognitive and motor function. There were no significant differences in behavior performance between these two dose groups.

### Example 4

### Longevity Assessment

Following the rAAV9-hNaGlu vector injection(s), mice were continuously observed for the development of endpoint symptoms, or until death occurred. The endpoint was when the symptoms of late stage clinical manifestation (urine retention, rectal prolapse, protruding penis) in MPS IIIB mice became irreversible, or when wt control mice were 24 months or older. Longevity data were analyzed using Kaplan-Meier method. The significance level was set at P<0.05. Results are shown in FIG. 2c.

Ten rAAV9-treated MPS IIIB mice, five from each dose group, were observed for longevity. All ten survived >16.9 months (with one mouse of the low-dose group dying at age of 16.1 months) and the majority of them survived 18.9-27.4 months within the normal range of lifespan, while all non-treated MPS IIIB mice died at 8-12 months of age (P<0.001) (FIG. 2c). These data demonstrate that a single IV rAAV9 vector injection alone is functionally beneficial in treating the CNS disease and increasing longevity in MPS IIIB mice.

### Example 5

### Tissue Analyses

In the therapeutic studies above, tissue analyses were carried out at 6mo and 9mo post injection (pi). Mice were anesthetized with 2.5% Avertin before tissue collection. Brain, spinal cord and multiple somatic tissues were collected on dry ice or embedded in OCT compound and stored at −70° C., before being processed for analyses. Tissues were also processed for paraffm sectioning.

Tissue samples from scAAV9-GFP vector-treated mice were collected for analysis 4-5 weeks pi. The mice were anesthetized with 2.5% Avertin and then perfused transcardially with cold PBS (0.1M, pH7.4), followed by 4% paraformaldehyde in phosphate buffer (0.1M, pH7.4). The entire brain and spinal cord, as well as multiple somatic tissues (including liver, kidney, spleen, heart, lung, intestine and skeletal muscles), were collected and fixed in 4% paraformaldehyde overnight at 4° C. before being further processed for vibratome sectioning.

### Naglu Activity Assay

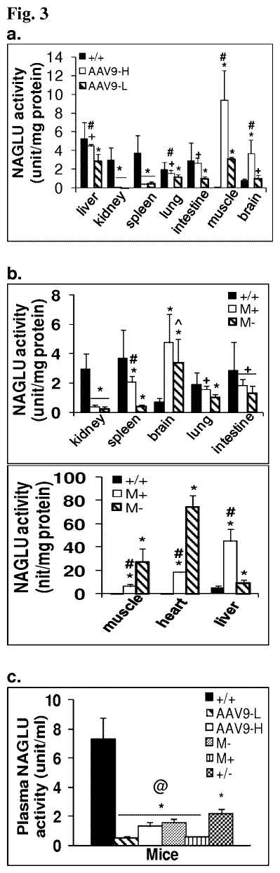

Tissues were analyzed at 6mo and/or 9mo pi by NAGLU activity assay to determine the distribution and level of rAAV9-mediated transgene expression. Tissue samples were assayed for NaGlu enzyme activity following a published procedure with modification. The assay measures 4-methylumbelliferone (4MU), a fluorescent product formed by hydrolysis of the substrate 4-methylumbellireyl-N-acetyla-D-glucosaminide. The NaGlu activity is expressed as unit/mg protein. 1 unit is equal to 1 nmol 4MU released/h at 37° C. Results are shown in FIG. 3.

### Gag Content Measurement

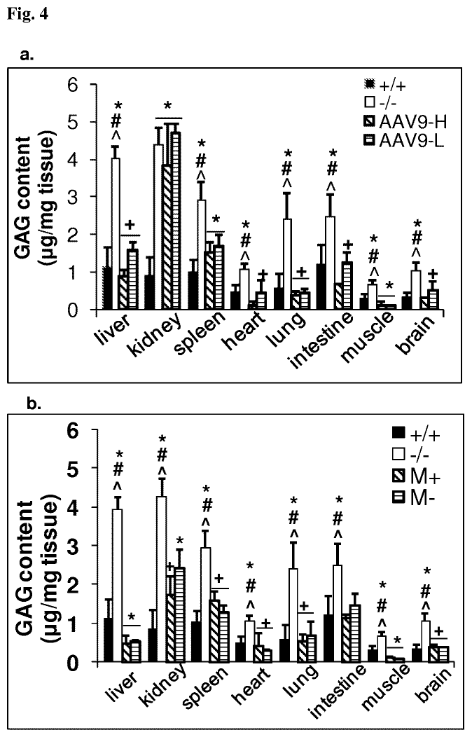

GAG was extracted from tissues following published procedures [van de Lest et al., Anal. Biochem. 221: 356-361(1994)] with modification [Fu et al., Gene Ther., 14: 1065-1077 (2007). Dimethylmethylene blue (DMB) assay was used to measure GAG content [de Jong et al., Clin. Chem., 35: 1472-1477 (1989)]. The GAG samples (from 0.5-1.0 mg tissue) were mixed with H₂O to 40 ml before adding 35 nM DMB (Polysciences CEO 03610-1) in 0.2 mM sodium formate buffer (SFB, pH 3.5). The product was measured using a spectrophotometer (0D535). The GAG content was expressed as μg/mg tissue. Urine GAG content was also measured. Heparan sulfate (Sigma, H9637) was used as standard. Results are shown in FIG. 4.

### Immunofluorescence

Immunofluorescence (IF) was performed to identify cells expressing hNAGLU, GFP or glial fibrillary acidic protein (GFAP) for astrocytes, using antibodies against hNaGlu (a kind gift from Dr. EF Neufeld, UCLA), GFP (Invitrogen) or GFAP (Chemicon), and corresponding secondary antibody conjugated with AlexaFluor⁵⁶⁸ or AlexaFluor⁴⁸⁸ (Molecular Probes). The IF staining was performed on thin cryostat sections (8p.m) of tissue samples following procedures recommended by the manufacturers. The sections were visualized under a fluorescence microscope.

### Histopathology

Tissues were assayed for histopathology to visualize the impact of IV rAAV9-NAGLU gene delivery on the lysosomal storage pathology in MPS IIIB mice. Histopathology was performed following standard methods. Paraffm sections (41.un) were fixed with 4% paraformaldehyde in phosphate buffer (0.1 M, pH 7.2) at 4° C. for 15 min and stained with 1% toluidine blue at 37° C. for 30 min to visualize lysosomal GAG. The sections were mounted, and imaged under a light microscope.

### Quantitative Real Time PCR

Total DNA was isolated from tissue samples of treated and nontreated MPS IIIB mice using Qiagen DNeasy columns. Brain DNA was isolated from midbrain tissue. The DNA samples were analyzed by quatitative real-time PCR, using Absolute Blue QPCR Mix (Thermo Scientific, Waltham, MA) and Applied Biosystems 7000 Real-Time PCR System, following the procedures recommended by the manufacturer. Taqman primers specific for the CMV promoter were used to detect rAAV vector genomes: f: GGCAGTACATC AAGTGTATC (SEQ ID NO: 6); r: ACCAATGG TAATAG CGATGAC (SEQ ID NO: 7); probe: [6˜FAM]AATGACGGTAAAT GGCCCGC[TAMRA˜6˜FAM](SEQ ID NO: 8). Genomic DNA was quantified in parallel samples using β-actin specific primers: f: GTCATCAC TATTG GCAACGA (SEQ ID NO: 9); r: CTCAGGAGTTTTGTCACCTT (SEQ ID NO: 10); probe: [6-FAM]TTCCGATGCCCT GAGGCTCT[Tamra-Q](SEQ ID NO: 11). Genomic DNA from nontreated MPS IIIB mouse tissues was used as controls or background and absence of contamination.Global CNS and widespread somatic restoration of NAGLU.

### Tissue Analysis Results

Tissues were analyzed at 6 months and/or 9 months pi by immunofluorescence (IF) and NAGLU activity assay to determine the distribution and level of rAAV9-mediated transgene expression. NAGLU-specific IF was detected throughout the brains of treated mice, in neurons, glia, and abundant endothelial cells in capillaries and larger blood vessels, in an apparently dose-dependent fashion. No significant differences were observed in the distribution or levels of rNaGlu signal between 6 months and 9 months pi. NAGLU-positive glial cells were not costained with anti-glial fibrillary acidic protein (GFAP) Ab, and were likely to be oligodendrocytes, based on their morphology. Importantly, while rNAGLU IF was observed in the brains of all rAAV9-treated mice, mannitol pretreatment did appear to increase the number of transduced cells in the CNS.

Differential transduction levels were observed in peripheral organs. The rNAGLU protein was detected in 20-40% of hepatocytes, >95% of cardiomyocytes, and 10-30% of skeletal myocytes. The distribution of rAAV9-transduced hepatocytes was uniform throughout the liver. Transduction in abundant neurons in myenteric plexus and submucosal plexus of the intestine was observed, suggesting efficient targeting of the peripheral nervous system (PNS). The rNAGLU signals were mostly present in granules, whereas scAAV9-mediated GFP signals were uniform in the cytoplasm of transduced cells, suggesting correct lysosomal trafficking of rNAGLU. Transduction of endothelial cells was also observed in peripheral tissues of rAAV9-GFP vector-treated mice.

### Example 6

### Enzyme Function Assays

Function of the recombinant NAGLU and resulting effects in animals were also analyzed in the therapeutic studies above.

rNAGLU Enzymatic Function

Transgene enzymatic activity was assayed to quantify the expression and the functionality of rAAV9-mediated rNAGLU. There were no significant differences in tissue NAGLU activity at 6 and 9 months pi, suggesting stable transduction. The rAAVmediated rNaGlu was metabolically functional and the tissue rNAGLU activity was dose-dependent, with approximately normal levels in the brains of mice receiving 5×10¹² vg/kg vector, and supra-physiologic levels in the brains of mice receiving 1.5×10¹³ vg/kg (FIG. 3a). In both dose groups, we detected NAGLU activity at normal or subnormal levels in the liver, lung and intestine (FIG. 3a), supra-physiologic levels in the skeletal muscles (FIG. 3a) and heart (40 & 100 units/mg protein, data not shown), and low levels in the spleen, but no detectable NAGLU activity in the kidney. A low level of NAGLU activity was detected in the kidneys of the mice treated with 2×10³ vg/kg vector (FIG. 3b). Mannitol pretreatment led to an increase in NAGLU activity in the brain (though not significant due to high individual variation), liver, spleen, lung and intestine, but a decrease in the heart and skeletal muscle (FIG. 3b). No detectable NAGLU activity (<0.03 unit/mg) was observed in tissues from non-treated MPS IIIB mice.

rNaGlu Secretion

Plasma samples were assayed for NAGLU activity to assess the secretion of the enzyme. Activity was detected in the plasma of all rAAV9-treated MPS MB mice at or near heterozygote levels, though lower than homozygous wt levels (FIG. 3c). Mannitol pretreatment resulted in significant reduction in plasma NAGLU activity (FIG. 3c). These data indicate that the rNAGLU was secreted, though the source tissue or cell type is not clear.

### GAG Content Reduction

Tissues were assayed for GAG content to quantify the impact of IV rAAV9-NAGLU gene delivery on the lysosomal storage pathology in MPS IIIB mice. The single IV rAAV9-NAGLU injection led to a reduction of GAG content to normal levels in the brain, liver, heart, lung, intestine and skeletal muscle in mice of all four treatment groups (FIG. 4). Doses of 5×10¹² g or 1.5×10³ vg/kg resulted in partial GAG reduction in the spleen but had no impact in kidney (FIG. 4a).

Treatment with 2×10³ vg/kg led to a decrease of GAG to normal levels in the spleen, and partial GAG reduction in the kidney (FIG. 4b), consistent with the observed enzyme activity levels.

### Histopathology Correction

Histopathology showed complete clearance or reduction of lysosomal storage lesions in the vast majority of CNS areas, including cerebral cortex, thalamus, brain stem, hippocampus, and spinal cord in all four treatment groups. There were decreases in the size, number of vacuoles, and number of cells with lysosomal storage lesions, even in the few brain areas that did not show a complete correction, such as purkinje cells and cells in the striatum and hypothalamus. Importantly, the majority of brain and spinal cord parenchymal cells exhibited a well defined normalized morphology. Immunofluorecence detection for the lysosomal marker, LAMP-1, showed that IV infusion of rAAV9-NAGLU vector at all doses also resulted in marked reduction of LAMP-1 signal, especially in neurons, throughout the brain. This further supports the conclusion that the amount of vector crossing the BBB was sufficient for efficient correction of CNS lysosomal storage pathology.

In somatic tissues, complete clearance of lysosomal storage lesions in the livers of all rAAV9-hNaGlu treated mice was observed as well as attenuation of nuclear shrinkage, a marker of cell stress and damage.

### Correction of Gliosis and Neurodegeneration

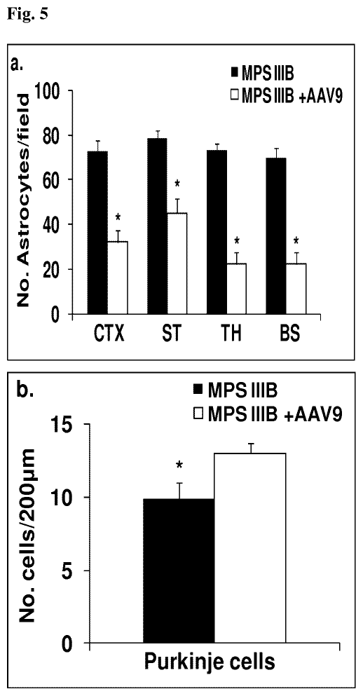

In order to determine whether the rAAV9-hNaGlu vector delivery had an impact on astrocytosis, a major secondary neuropathology of MPS IIIB, brain sections were assayed by immunofluorecence for GFAP expressing cells. Significant decreases in astrocyte numbers in gray matter throughout the brain of treated mice were observed compared to untreated at 6mo and 9mo pi (FIG. 5a). Histopathology also revealed significant increases in the numbers of neurons, such as Purkinje cells (FIG. 5b), in the brains of treated MPS IIIB mice. These data strongly indicate the amelioration of astrocytosis and neurodegeneration, which are hallmarks of secondary neuropathologies in MPS IIIB, in response to the rAAV9 treatment.

### Vector Genome Distribution

Quantitative real-time PCR was performed to compare the amount of rAAV9-CMV-hNaglu vector entering the CNS versus somatic tissues. Table 1 shows the distribution of the vector genome in different tissues/organs of MPS IIIB mice treated with IV vector injection at varying doses. The highest concentrations of vector genome were detected in liver (8.20±4.73-32.09±3.93 copies/cell), followed by heart (0.07-0.22 copies/cell), and brain (0.06±0.001-0.15±0.02 copies/cell), and very low copy numbers were detected in other tissues/organs (Table 1). This differential vector distribution in rAAV9-treated MPS IIIB mice largely correlated with the distribution of rNAGLU IF and enzymatic activity. Notably, mannitol pretreatment increased the vector copy numbers in the brain, correlating with brain NAGLU activity levels. Furthermore, these data reflect persistence of vector genome distribution in treated mice at 6 months pi, supporting a stable long-term transduction. Levels of vector genome copies correlating with rNAGLU activity and distribution were not detectable, possibly due to difficulties in quantitative isolation of DNA from muscle tissue.

TABLE 1
Estimated vector genome in the liver
and brain of rAAV9-treated mice

| | | Vector genome (copy/cell) | | |
|---|---|---|---|---|
| Mice | n | Liver | Brain | Heart |
| rAAV9-L | 2 | 8.20 ± 4.73 | 0.07 ± 0.07 | 0.07* |
| rAAV9-H | 3 | 10.86 ± 2.94 | 0.09 − 10.47 | 0.13 ± 0.07 |
| rAAV9-M− | 2 | 21.97 ± 6.43 | 0.06 ± 0.001 | 0.22* |
| rAAV9-M+ | 3 | 32.09 ± 3.93 | 0.15 ± 0.02 | 0.14* |
| Non-treated | 1 | 0.000 | 0.000 | 0.00 |

Mouse tissue samples (6 mo pi) were assayed in duplicates for vector genome copy numbers by qPCR. Data is expressed as vector copy/cell (means ± SD). rAAV9-L: IV infusion of 5 × 10¹² vg/kg; rAAV9-H: IV infusion of 1.5 × 10¹³ vg/kg; rAAV9-M−: IV infusion of 2 × 10¹³ vg/kg without mannitol pretreatment; rAAV9-M+: IV infusion of 2 × 10¹³ vg/kg following mannitol pretreatment.
*Data from 1 sample in duplicates.

### Example 7

### Discussion

This study demonstrates the first significant therapeutic benefit for treating MPS IIIB in adult animals from systemic gene delivery to the CNS without additional treatment to disrupt the BBB. A single IV injection of hNAGLU-expressing rAAV9 vector was sufficient to significantly improve cognitive and motor functions, and greatly prolong survival in MPS IIIB mice. In the present study using rAAV9, the increased longevity exceeds the outcome of previous studies using rAAV2 vector delivered through either intracisternal injection, or systemic injection following mannitol pretreatment. The rNAGLU enzyme was clearly secreted and functional, leading to a significant bystander effect, and efficient degradation of heparan sulfate GAG in CNS tissues. Importantly, the clinically meaningful therapeutic benefits of the IV-delivered rAAV9 vector in MPS IIIB mice were achieved at a lower dose than the mannitol-facilitated, systemically delivered rAAV2 vector. The enhanced rAAV9-CNS transduction in response to mannitol pretreatment suggests further potential for reducing the vector dose, and the attendant risk and burden to patients.

The IV vector injection resulted in a ubiquitously diffuse, global rAAV9-NaGlu transduction throughout the CNS, reflecting the expected distribution pattern for vascular delivery. This contrasts sharply with the focal gradient distribution typically achieved through direct brain parenchymal injection, or the periventricular transduction pattern from intracisternal and intraventricular injection. While similar to the pattern of transgene expression from IV-delivered rAAV2 after mannitol pretreatment, the extent of rAAV9 transduction was significantly higher in all areas of the brain. This correlates with the increased effects on longevity and cognitive function compared to that previously achieved using rAAV2-mannitol treatment, and the normal or above normal levels of NAGLU activity in the CNS. These findings strongly support the use of the trans-BBB neurotropic rAAV9 as a vector for CNS gene therapy and reinforce the view that efficient CNS delivery is the most critical issue for developing therapies to treat MPS IIIB.

The rAAV9-transduced CNS cells include neurons, glia and endothelia. Neuronal cell transduction appears to be non-preferential, including most types of neurons throughout the brain. In contrast, the transduction of glial cells appears to be cell-type specific, targeting predominantly oligodendrocyte-like cells, though it is unclear whether this is a receptor- or promoter-specific phenomenon. In a previous report [Faust et al., supra] describing predominant transduction of astocytes after systemic injection of rAAV9 vector in adult mice, a hybrid chicken J3-actin/CMV-enhancer promoter was used, rather than the CMV enhancer-promoter used in the present study.

In normal cells, 5-20% of newly synthesized lysosomal protein is secreted and available to be taken up by neighboring cells, leading to the by-stander effect. The widespread clearance/reduction of lysosomal storage pathology, and normalized tissue GAG content, strongly support an efficient by-stander effect from the rAAV9¬mediated rNAGLU. The abundant transduction of endothelial cells in the brain may be an important contributor to the effectiveness of rAAV9 gene delivery for MPS IIIB because of the close association between CNS cells and brain microvascular endothelial cells, which together constitute the neurovascular unit. While the observed high levels of rNAGLU expression stem from the transduction of a relatively small number of CNS cells, it is sufficient to correct the neuropathology leading to functional correction of the neurological disorders.

The rAAV9 treatment also led to a regular morphology in CNS cells, and the correction of major secondary neuropathology, astrocytosis, and neurodegeneration. It is worth noting that this level of correction of CNS pathology was not achieved in previous studies using rAAV2-hNAGLU vector with mannito1. While neuropathology is the primary cause of mortality in MPS IIIB patients, somatic correction may provide additional therapeutic benefits, since lysosomal storage pathology inevitably manifests in virtually all organs. The IV-delivered rAAV9 exhibited broad tropism in peripheral tissues in a distinct pattern, as previously reported, reflecting extensive extravasation and cell-type specific transduction. This led to complete, long¬term correction of lysosomal storage in multiple somatic tissues even at a relatively low dose. Again, relatively low levels of transduction in some tissues were associated with clearance of lysosomal storage of GAGs in the organs, consistent with a significant contribution from the by-stander effect of secreted rNAGLU enzyme. It is not clear whether the by-stander correction in peripheral tissues is mediated by enzyme secreted from neighboring cells within the same tissue, or circulating rNAGLU secreted by more extensively transduced tissues, in a manner analogous to enzyme replacement therapy. However, the observation of partial GAG reduction in the kidney only at the highest vector dose, correlating with detectable transduction in the kidney only at that dose, suggests that the by-stander effect may be primarily local in this tissue. The primary source of circulating NAGLU may be liver, muscle, or endothelium. However, the decrease in plasma levels in response to mannitol pretreatment correlated with decreased transduction in muscle rather than liver, suggesting that liver may not be the primary source.

Another important observation is the efficient transduction of neurons in myenteric plexus and submucosal plexus of the intestine, potentially enabling correction of not only the CNS but also the PNS at all levels via systemic delivery. This suggests that neurotropism is a general property of the AAV9 serotype, and not dependent on the specific structure of the brain neurovascular unit. Broad neurotropism is a valuable property in gene therapy for the treatment of MPS IIIB, considering that lysosomal storage pathology manifests not only in the CNS but also in the PNS.

### Example 8

### Recombinant AAV (rAAV) Viral Vectors Encoding SGSH

A rAAV vector plasmid was used to produce three different rAAV9-CMV-hSGSH viral vectors.

The three self-complementary AAV hSGSH vector-producing plasmids were constructed using conventional plasmid cloning techniques. Each vector genome contains an SGSH coding region (SEQ ID NO: 3) and either the mouse U1a promoter, with or without an intron, or a CMV promoter without an intron, Each vector genome also contains a bGH polyadenylation signal. Each self-complementary vector plasmid construct contains one intact AA2 terminal repeat and one modified AAV2 terminal repeat missing the terminal resolution site, thereby forcing the replication of dimeric self-complementary DNA genomes. Self-complementary AAV hSGSH viral vectors were produced and packaged in AAV serotype 9 capsids. The viral vectors were tested for expression of hSGSH protein and reduction of GAG storage in human MPS IIIA fibroblasts.

Self-complementary AAV hSGSH viral vectors were tested in an MPS IIIA mouse model having a missense mutation in the SGSH gene [Bhaumik et al., Glycobiology, 9(12):1389-1396 (1999)] as described in the examples below.

### Example 9

MPS IIIA mice were injected at 10 weeks of age with 5×10¹² vgp/kg) of scAAV-U1a-hSGSH vector encapsidated in either AAV9 or AAVrh74 serotype. At 10 days post-injection, the mice were euthanized and assays were performed to determine the effects of the treatment.

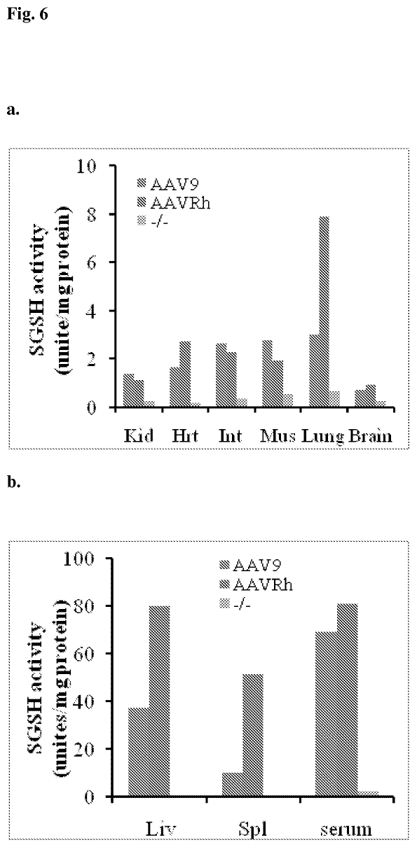

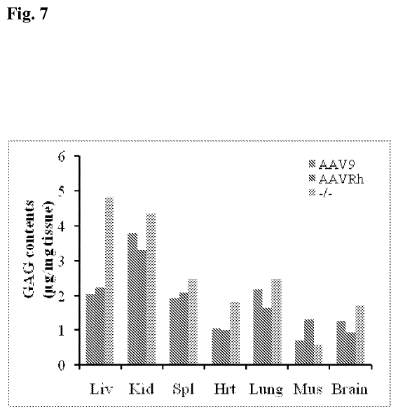

hSGSH transgene expression was assayed. Tissues analyzed included kidney (Kid), heart (Hrt), intestine (Int), skeletal muscle (Mus), lung, brain, Liver (Liv), spleen (Spl) and serum. FIG. 6 shows enzyme expression relative to untreated MPS IIIA mice at the same age (−/−). The scAAV-SGSH vectors reached the CNS and expressed the transgene within days of administration. FIG. 7 shows GAG content measured in the kidney (Kid), heart (Hrt), muscle (Mus), lung, brain, Liver (Liv) and spleen (Spl).

Sections of CNS and somatic tissues were stained with the lysosomal marker, Lamp1, revealing clearance of lysosomal storage pathology. Histopathology additionally revealed numerous clear vacuoles present in untreated mice but corrected in treated animals.

### Example 10

The therapeutic effects of scAAV-SGSH treatment at a low vector dose were examined.

Vector was administered by tail vein injection in MPS IIIA mice at one month of age at an approximately 25-fold lower dose than in Example 9. MPS IIIA mice were treated with 1.7×10¹¹ vgp/kg scAAV9-U1a-hSGSH or 2.7×10¹¹ vgp/kg scAAVrh74-U1a-hSGSH vector.

At three months post-injection, expression of SGSH in the CNS was observed by immunofluroescence staining. Correction of astrocytocis, a hallmark of neuroinflamation associated with MPS IIIA pathology, was also observed.

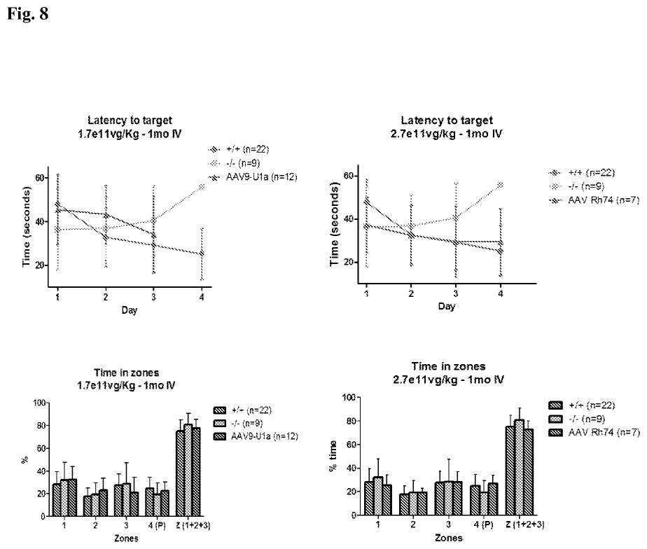

At 7-7.5 months age, the animals were tested for learning ability in the Morris water maze. As shown in FIG. 8, compared to untreated controls, treated animals were similar to wt mice in their latency to locate the hidden platform (upper charts) and spent more time in the zone (4) where the platform had been in the previous tests when the platform was removed (lower charts).

### Example 11

Therapeutic effects of scAAV treatment at high dose at late stage of disease were also examined.

MPS IIIA mice were treated with a high dose (5×10¹² vgp/kg) of scAAV9-U1a-hSGSH vector at 6 months of age, after significant neuropathology had already developed. At 7-7.5 months age, the animals were tested for learning ability in the Morris water maze. At 7.5 months of age, the mice were euthanized and tissues assayed for glycosaminoglycan (GAG) content. Tissues analyzed include liver (Liv), kidney (Kid), heart (Hrt), brain, spleen (Spl), lung, skeletal muscle, and intestine.

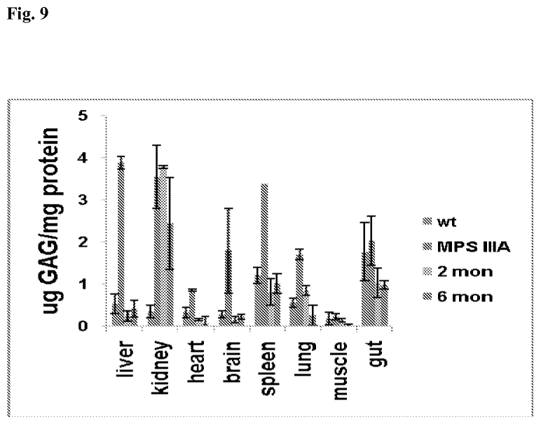

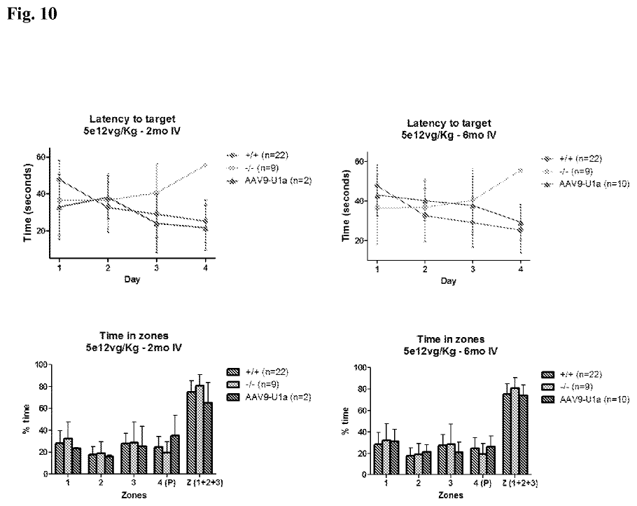

FIG. 9 shows clearance of accumulated GAGs in different tissues, including CNS. FIG. 10 shows, compared to untreated controls, treated animals were similar to wt mice in their latency to locate the hidden platform (upper charts) and spent more time in the zone (4) where the platform had been in the previous tests when the platform was removed (lower charts).

While the present invention has been described in terms of various embodiments and examples, it is understood that variations and improvements will occur to those skilled in the art. Therefore, only such limitations as appear in the claims should be placed on the invention.

All documents referred to in this application are hereby incorporated by reference in their entirety.

1-31. (canceled)

32. A recombinant adeno-associated virus (rAAV) for systemically delivering N-sulphoglucosamine sulphohydrolase (N-SGSH) to the central and peripheral nervous systems of a subject with mucopolysaccharidosis IIIA (MPSIIIA), the rAAV comprising an AAV capsid and a self-complementary rAAV genome packaged therein, the self-complementary rAAV genome comprising:
a polynucleotide sequence that has at least 99% sequence identity to the polynucleotide sequence consisting of SEQ ID NO: 3 encoding a biologically active N-SGSH polypeptide;
a promoter;
a polyadenylation signal; and
at least one AAV terminal repeat.

33. The rAAV of claim 32, wherein the promoter is a U1a promoter.

34. The rAAV of claim 32, wherein the promoter is a CMV promoter.

35. The rAAV of claim 32, wherein the rAAV genome further comprises an intron.

36. The rAAV of claim 32, wherein the polyadenylation signal comprises a bGH polyadenylation signal.

37. The rAAV of claim 32, wherein the polyadenylation signal comprises an SV40 polyadenylation signal.

38. The rAAV of claim 32, wherein the at least one AAV terminal repeat comprises at least one AAV2 terminal repeat.

39. The rAAV of claim 38, wherein the at least one AAV2 terminal repeat comprises an intact AAV2 terminal repeat and/or a modified AAV2 terminal repeat missing the terminal resolution site.

40. A composition comprising the rAAV of claim 32 and a pharmaceutically acceptable carrier.

41. The composition of claim 40, wherein the composition is suitable for intravenous injection to the subject.

42. The composition of claim 41, wherein the composition is suitable for intravenous injection to the subject at a dose of about 1.0×10¹³ vg/kg.

43. The composition of claim 41, wherein the composition is suitable for intravenous injection to the subject at a dose of about 3.0×10¹³ vg/kg.

44. The composition of claim 41, wherein the composition is suitable for intravenous injection to the subject at a dose of about 1.0×10¹⁴ vg/kg.

45. The composition of claim 41, wherein the composition is suitable for intravenous injection to the subject at a dose of about 1.0×10¹⁵ vg/kg.

46. A recombinant polynucleotide comprising the rAAV genome of claim 32.

47. A packaging cell comprising the recombinant polynucleotide of claim 46 and AAV rep and cap genes.

48. The packaging cell of claim 47, wherein the packaging cell is a HEK293 cell.

49. The packaging cell of claim 47, wherein the packaging cell is a HeLa cell.

50. A method of producing a recombinant adeno-associated virus (rAAV) encoding N-sulphoglucosamine sulphohydrolase (N-SGSH), the method comprising
providing to a cell a rAAV genome to be packaged, AAV rep and cap genes, and helper virus functions, wherein the rAAV genome comprises:
(i) a polynucleotide sequence that has at least 99% sequence identity to the polynucleotide sequence consisting of SEQ ID NO: 3 encoding a biologically active N-SGSH polypeptide;
(ii) a promoter;
(iii) a polyadenylation signal, and
(iv) at least one AAV terminal repeat.

51. The method of claim 50, wherein the rAAV genome and the AAV rep and cap genes are stably integrated in the cell, and wherein providing helper virus functions to the cell comprises infecting the cell with adenovirus.
